(19) United States
(12) Patent Application Publication
(10) Pub. No.: US 2026/0335110 A1
(43) Pub. Date: Oct. 29, 2026

(54) DEVICES, SYSTEMS AND METHODS FOR ACCESS AND DELIVERY TO CHALLENGING ANATOMY

(71) Applicant: Boston Scientific Scimed, Inc., Maple Grove, MN (US)

(72) Inventors: Martin Burke, Loughrea (IE); Gerard Duignan, Loughrea (IE); Louis McNern, Kinvara (IE); David Collins, Athenry (IE); Kevin McEvilly, Oranmore (IE); Scott Stephen Lueders, Medfield, MA (US); Orlaith Marie Duffy, Claremorris (IE); Meredith Margaret Dixon, Galway (IE)

(73) Assignee: Boston Scientific Scimed, Inc., Maple Grove, MN (US)

(21) Appl. No.: 19/661,281

(22) Filed: Apr. 28, 2026

Related U.S. Application Data
(60) Provisional application No. 63/796,222, filed on Apr. 28, 2025.

(51) Int. Cl. A61F 2/95; A61F 2/966

(52) U.S. Cl. CPC A61F 2/9517; A61F 2/966

(57) ABSTRACT

Delivery devices, systems and procedures are disclosed for delivering an implant, such as a stent. The delivery device may include an outer sheath, an inner member, and a handle. The outer sheath may have a proximal end, a distal end, and a lumen extending longitudinally therethrough. The inner member may be disposed within the lumen of the outer sheath. The handle may include a body, a puller member coupled with the proximal end of the outer sheath, a pusher member comprising a connector and coupled with the proximal end of the inner member, and a stopper coupled with the body and positioned between the puller member and the pusher member. Adjustment of the pusher member toward the stopper may adjust the inner member relative to the outer sheath and may cause the connector to engage the stopper and prevent proximal movement of the pusher member relative to the stopper.

### CROSS-REFERENCE TO RELATED APPLICATIONS

This application claims the benefit of U.S. Provisional Patent Application Ser. No. 63/796,222 filed on Apr. 28, 2025, the disclosure of which is incorporated herein by reference.

### TECHNICAL FIELD

The disclosure pertains to medical delivery devices, systems and methods, and more particularly to delivery devices for delivering implants such as stents into challenging anatomy such as confined or limited spaces in the body.

### BACKGROUND

A wide variety of intracorporeal and extracorporeal medical devices and systems have been developed for medical use, for example, for endoscopic procedures. Some of these devices and systems include guidewires, catheters, catheter systems, endoscopes, ablation devices, delivery systems, stents, anchors, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices, including stent delivery devices, systems, and methods. In one example, a delivery device may include an outer sheath having a proximal end, a distal end, and a lumen extending longitudinally therethrough, an inner member disposed within the lumen of the outer sheath, and a handle comprising a body, a puller member coupled with the proximal end of the outer sheath, a pusher member comprising a connector and coupled with the proximal end of the inner member, and a stopper coupled with the body and positioned between the puller member and the pusher member, wherein adjustment of the pusher member toward the stopper may adjust the inner member relative to the outer sheath and causes the connector to engage the stopper and prevent proximal movement of the pusher member relative to the stopper.

Alternatively or additionally to any of the examples above, the puller member may be configured to be adjusted toward the stopper to adjust the outer sheath relative to the inner member and engage the connector to release the connector from engagement with the stopper.

Alternatively or additionally to any of the examples above, the connector may comprise a tapering distal surface and the puller member may be configured adjust relative to the stopper to engage the tapering distal surface to release the connector from engagement with the stopper.

Alternatively or additionally to any of the examples above, the pusher member may have a base and the connector is configured to extend in a distal direction from the base.

Alternatively or additionally to any of the examples above, the connector may be configured to be biased to a first position and flex to a second position relative to the base.

Alternatively or additionally to any of the examples above, the connector may comprise a protrusion configured to engage a distal facing surface of the stopper.

Alternatively or additionally to any of the examples above, the stopper may comprise a lock, the lock may be configured to adjust from a first position in which the pusher member is able to adjust longitudinally relative to the stopper and a second position in which the pusher member is longitudinally fixed relative to the stopper.

Alternatively or additionally to any of the examples above, the handle may further include an actuator disposed within a lumen of the body, wherein adjustment of the actuator relative to the body longitudinally may adjust the outer sheath and the inner member relative to the body.

Alternatively or additionally to any of the examples above, the delivery device may further include a distal tip disposed at a distal end of the inner member, wherein the distal tip may be distal of the outer sheath when the inner member is fully advanced in a proximal direction relative to the outer sheath.

In another example, a system may include a delivery device including an outer sheath having a proximal end, a distal end, and a lumen extending longitudinally therethrough, an inner member disposed within the lumen of the outer sheath and a handle comprising a body, a puller member coupled with the proximal end of the outer sheath, a pusher member coupled with the proximal end of the inner member, the pusher member comprising a connector, and a stopper coupled with the body and positioned between the puller member and the pusher member, and a self-expanding stent constrained on the inner member between the inner member and the outer sheath and deployable therefrom by first pushing the inner member with the pusher member relative to the outer sheath and then pulling the outer sheath with the puller member relative to the inner member, wherein pushing inner member with the pusher member causes the connector to engage the stopper and prevent proximal movement of the inner member relative to the outer sheath.

Alternatively or additionally to any of the examples above, the puller member may be configured to engage the connector to release the connector from engagement with the stopper and allow proximal movement of the inner member relative to the outer sheath.

Alternatively or additionally to any of the examples above, when the self-expanding stent is fully deployed, the self-expanding stent may be shaped and sized such that a distal tip of the inner member can be retracted proximally through the self-expanding stent.

Alternatively or additionally to any of the examples above, the connector may include a tapering distal surface and the puller member is configured to engage the tapering distal surface to release the connector from engagement with the stopper.

Alternatively or additionally to any of the examples above, the connector may include a protrusion configured to engage a distal facing surface of the stopper.

Alternatively or additionally to any of the examples above, the stopper may include a lock, the lock may be configured to adjust from a first position in which the pusher member is able to adjust longitudinally relative to the stopper and a second position in which the pusher member is longitudinally fixed relative to the stopper.

In another example, a method of using a stent delivery device may comprise positioning a distal tip of a delivery device at a location proximate a target location, the delivery device carrying a stent at a location proximate the distal tip and between an inner member and an outer sheath of the delivery device, deploying a distal end of the stent from the delivery device by advancing a pusher member of the delivery device toward a stopper of the delivery device until a connector of the pusher member couples with the stopper to prevent movement of the pusher member away from the stopper, and with the distal end of the stent deployed, deploying a proximal end of the stent from the delivery device by advancing a puller member of the delivery device toward the stopper until the puller member engages the connector and causes the connector to disengage from the stopper.

Alternatively or additionally to any of the examples above, the method may include after the connector is disengaged from the stopper, withdrawing the distal tip of the delivery device to a location proximate a distal end of the outer sheath by advancing the pusher member in a direction away from the stopper.

Alternatively or additionally to any of the examples above, the method may include with the distal tip at the location proximate the distal end of the outer sheath, withdrawing the distal tip of the delivery device in a proximal direction from the location proximate the target location.

Alternatively or additionally to any of the examples above, the method may include visualizing the distal tip of the delivery device as the distal tip is withdrawn in the proximal direction from the location proximate the target location.

Alternatively or additionally to any of the examples above, positioning the distal tip of the delivery device at the location proximate the target location may include advancing an actuator of the delivery device relative to a handle body of the delivery device.

The above summary of some configurations is not intended to describe each disclosed configuration or every implementation of the present disclosure. The Figures, and Detailed Description, which follow, more particularly exemplify some of these configurations.

### BRIEF DESCRIPTION OF THE DRAWINGS

The disclosure may be more completely understood in consideration of the following detailed description in connection with the accompanying drawings, in which:

FIG. 1 depicts a schematic view of an illustrative configuration of a delivery device;

FIG. 2 depicts a schematic perspective view of a handle body and a distal tip of an illustrative delivery device;

FIG. 3 depicts a schematic exploded view of a portion of the delivery device depicted in FIG. 2;

FIG. 4 depicts a schematic exploded view of a portion of the delivery device depicted in FIG. 2;

FIG. 5 depicts a schematic perspective view of a portion of the delivery device depicted in FIG. 2; and

FIGS. 6A-6G depict schematic views of steps in an illustrative method of using a delivery device.

While aspects of the disclosure are amenable to various modifications and alternative forms, specifics thereof have been shown by way of example in the drawings and will be described in detail. The intention, however, is not to limit aspects of the disclosure to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar structures in different drawings are numbered the same. The drawings, which are not necessarily to scale, depict illustrative configurations and are not intended to limit the scope of the disclosure. Additionally, it should be noted that in any given figure, some features may not be shown, or may be shown schematically, for clarity and/or simplicity. Additional details regarding some components and/or method steps may be illustrated in other figures in greater detail. The devices and/or methods disclosed herein may provide a number of desirable features and benefits as described in more detail below.

Illustrative stent delivery devices, systems and methods that may be used in endoscopic medical procedures are disclosed. The procedures may be to, for example, access a first organ or lumen by penetrating and creating an opening through a tissue wall of that organ or lumen and placing a stent to maintain the opening on a temporary or prolonged basis. Sometimes, access may include penetrating and creating an opening through a tissue wall of a second organ or lumen that has a tissue wall apposing or adjacent to the tissue wall of the first organ. A stent may be placed to span the openings in the walls with the apposed or adjacent walls engaged between retention members at each end of the stent. The access may be for establishing drainage of one organ into another, creating a path for further instrument access, creating an anastomosis between adjacent tissues walls, such as with a gastrojejunostomy procedure, and the like.

Reference to a gastrojejunostomy procedure is provided only for convenience and not intended to limit the disclosure. The concepts underlying the disclosed devices and related methods of use may be utilized in any suitable procedure, medical or otherwise. This disclosure may be understood with reference to the following description and the appended drawings. The same or similar reference numbers will be used throughout the drawings to refer to the same or like parts.

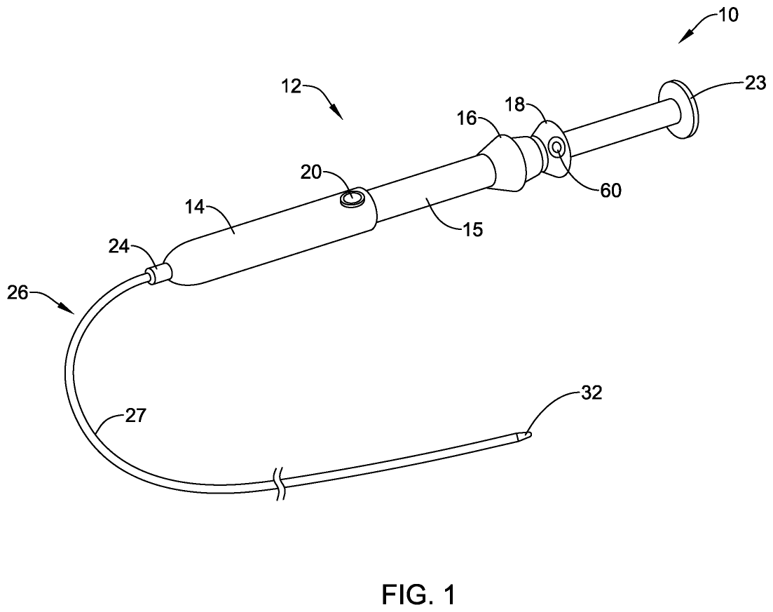

FIG. 1 schematically depicts an illustrative configuration of a delivery device 10. The delivery device 10 may include, among other components, a handle 12 having a body 14 with a first slide actuator 15, a knob 16, and a lock 20. In some examples, the delivery device 10 may include a second slide actuator 18 with a lock 60, a scope locking mechanism 24 (e.g., a scope lock), an electrical plug 23, catheter body 26, an outer sheath 27, and a distal tip 32. In some examples, the distal tip 32 may be attached to an inner shaft and may be slidable through the outer sheath 27. The outer sheath 27 may be attached to the second slide actuator 18 and may be slidable through the handle 12, such that moving the second slide actuator 18 in the proximal direction pulls the outer sheath 27 in the proximal direction relative to the inner shaft and the handle 12. The first slide actuator 15 may be connected to both the outer sheath 27 and the inner shaft, such that actuation of the first slide actuator 15 moves the inner shaft and outer sheath 27 relative to the body 14.

The lock 20 may be associated with the first slide actuator 15 and configured to lock the first slide actuator 15 relative to the body 14. Unlocking the lock 20 may allow a user to move the first slide actuator 15, moving the inner shaft and the outer sheath 27 together, to effect how far the inner shaft and the outer sheath 27 extend from a distal end of the body 14.

The lock 60 of the second slide actuator 18 may be configured to lock the second slide actuator 18 relative to the first slide actuator 15, the body 14, and inner shaft. Unlocking the lock 60 may allow the user to pull the second slide actuator 18 proximally relative to the first slide actuator 15, the body 14, and the inner shaft, deploying a distal flange of the stent. The second slide actuator 18 may then be fully retracted proximally to deploy a proximal end of the stent.

The distal tip 32 may include a conductive area, configured to cut, heat, and/or cauterize tissue in a patient. The distal tip 32 may be used to create or widen a hole in a tissue wall for the device to pass through prior to deploying the stent. Electrical energy may be supplied to the distal tip 32 in the form of radiofrequency (RF) energy, high frequency (HF) energy, and/or other suitable energy. The electrical energy may be supplied from a power source (e.g., an electrical generator, etc.) through the electrical plug 23.

The delivery device 10 may include or may be coupled with a stent (e.g., a self-expanding stent, etc.) that may be retained for deployment near the distal end of the delivery device 10 between in the inner shaft and the outer sheath 27. Deployment of the stent may occur upon proximal retraction of the outer sheath 27 to deploy a distal flange of the stent, and then proximal retraction of the outer sheath 27 to deploy a proximal flange of the stent. This deployment requires two pulling motions by the user, to retract the external sheath proximally, and release the stent from its retained position.

Due to the nature of self-expanding stents, stents can be prone to foreshortening. For example, stents, when constrained, may have a different linear length than when they are fully deployed, which may affect a deployed position of the stent. Foreshortening of the stent during delivery may pose an issue when the delivery location is in a small space, or close to a tissue wall or organ. The anatomy of a desired deployment location may prevent distal movement of the device to compensate for foreshortening of the stent when the stent is initially deployed with a proximal movement of an outer sheath. The two proximal retractions of the external sheath can make it difficult to adjust stent placement during the procedure, and may cause the stent to be placed too far proximally, at the wrong angle, and/or within the wrong body channel.

Illustrative configurations of the devices, systems, and methods discussed herein may facilitate phased deployment of a stent, including a distal push forward of an inner member of a device while an outer sheath remains stationary to deploy the distal end of the stent, and then a proximal pull backward of the outer sheath while the inner member remains stationary to deploy the proximal end of the stent. Such configurations may be advantageous and allow for more precise positioning and control over delivery of stents into challenging anatomy, such as small spaces, narrow lumens, spaces requiring a complicated angle of approach, lumens that are typically not-expanded when empty, etc.

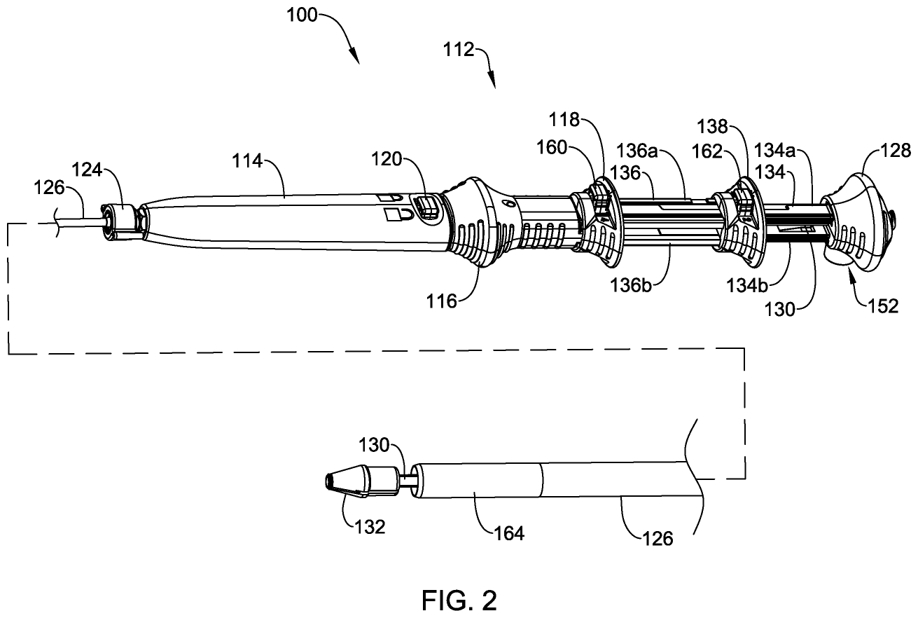

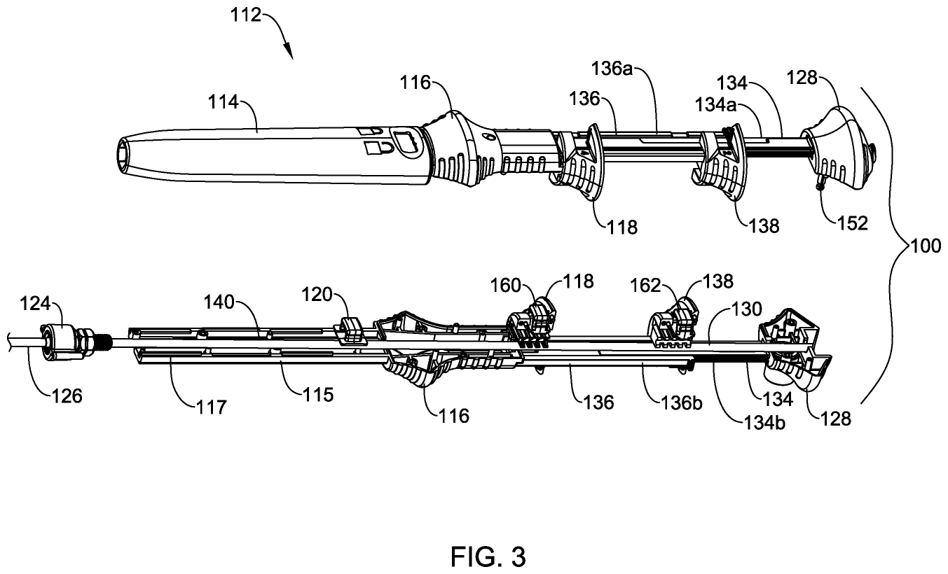

FIG. 2 schematically depicts a perspective view of an illustrative configuration of a delivery device 100, which may have some similar features to those discussed with respect to the delivery device 10 and/or other suitable features. In some examples, the delivery device 100 may include a pusher member 128, a puller member 118, and a stopper 138 located between the pusher member 128 and the puller member 118. In some examples, the delivery device 100 may include a handle 112 with a handle housing or body 114, a scope lock 124 (e.g., a luer lock and/or other suitable type of lock), a knob 116 attached to a handle actuator 115 (FIG. 3), and one or more locks (e.g., a first lock 120 locate on or at the handle body 114, a second lock 160 located on or at the puller member 118, and a third lock 162 located on or at the stopper 138). In some examples, the delivery device 100 may include an outer sheath 126 extending distally from the distal end of the handle body 114, and slideably passing through a lumen of the handle body 114 and the scope lock 124.

In addition to or as an alternative to the delivery device 100 including the stopper 138, any suitable element configured to limit movement of the outer sheath 126 and/or an inner member 130 of the delivery device 100 may be utilized, either as a separate element of the delivery device 100 or as an element or feature of one of the other components of the delivery device 100. In some examples, the stopper 138 between the puller member 118 and pusher member 128 may be omitted.

The inner member 130 may extend through a lumen of the outer sheath 126 and a distal tip 132 may be located at a distal end of the inner member 130. In some examples, the distal tip 132 may extend distal of a distal end of the outer sheath 126. The distal tip 132 and the inner member 130 may be a single monolithic element or may be separate elements joined together. In some examples, the inner member 130 may include a lumen through which a guidewire, fluid, or other component may pass through toward the distal tip 132. In some configurations, the distal tip 132 may be able to be retracted within the outer sheath 126. The distal tip 132 may include a conductive area, configured to cut, heat, and/or cauterize tissue in a patient in order to penetrate the tissue and create an opening therethrough. Electrical energy may be supplied to the distal tip 132 in the form of radiofrequency (RF) energy, high frequency (HF) energy, and/or other suitable types of energy. The electrical energy may be supplied from a power source (e.g., electrical generator, etc.) to the device through an electrical plug 152 at the pusher member 128 and/or at one or more other suitable locations.

The pusher member 128 may include a set of inner rails 134 (e.g., a first or upper inner rail 134a and a second or lower inner rail 134b), slidable along a set of outer rails 136 (e.g., a first or upper outer rail 136a and a second or lower outer rail 136b) attached to the handle actuator 115 (FIG. 3). The inner rails 134 and pusher member 128 may be a single monolithic element or may be two distinct elements joined together. The outer rails 136 and handle actuator 115 may be a single monolithic element or may be two distinct elements joined together. In some examples, the inner rails 134 and the outer rails 136 may have a tongue and groove arrangement, ridges, joints, or other suitable interlocking configured to maintain the pusher member 128 in place as the pusher member 128 and/or the inner rails 134 slide longitudinally along the outer rails 136. The inner member 130 may be attached to the pusher member 128 such that moving the pusher member 128 and inner rails 134 in the proximal or distal direction along the outer rails 136 causes movement of the inner member 130 in the proximal or distal direction relative to the handle actuator 115, the handle body 114, and//or the outer sheath 126. The pusher member 128 may also include a lock (e.g., luer lock, etc.) to allow for attaching a syringe, fluid pump, or the like to the proximal end of the handle 112.

The pusher member 128 may be configured to advance in the distal direction until the pusher member 128 reaches the stopper 138. The lock 162 of the stopper 138 may be configured to hold the pusher member 128 in place against the stopper 138 when the lock 162 is in a locked configuration. The stopper 138 may be fixedly attached to the proximal end of the outer rails 136 of the handle actuator 115 or integral therewith. The stopper 138 may include through channels that the inner rails 134 of the pusher member 128 may pass through as the inner rails 134 slide along the outer rails 136. The through channels of the stopper 138 may be useful to stabilize or guide the inner rails 134 as the inner rails 134 slide along the outer rails 136. Movement of the pusher member 128 may extend the inner member 130 and the distal tip 132 relative to and distally out of the outer sheath 126.

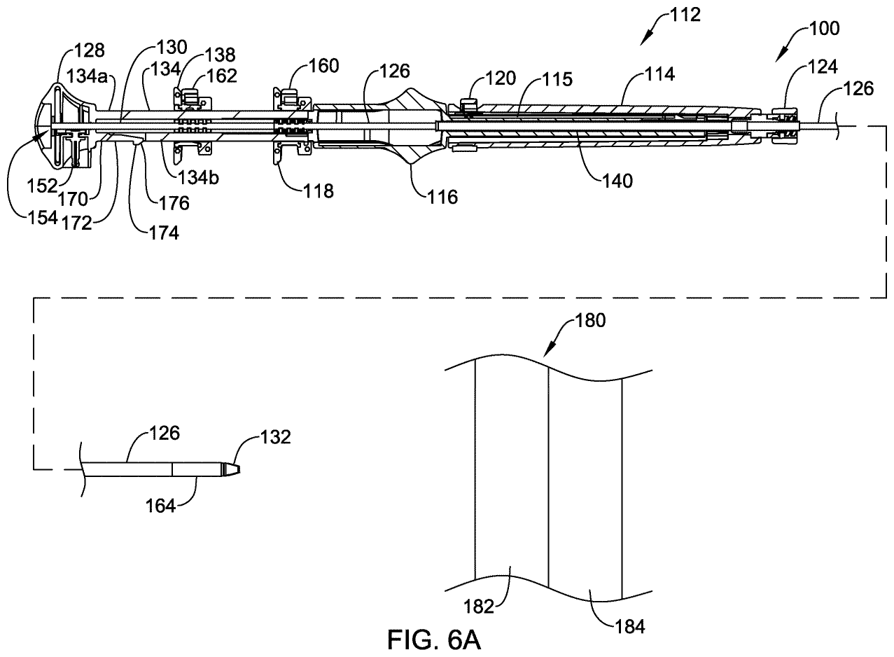

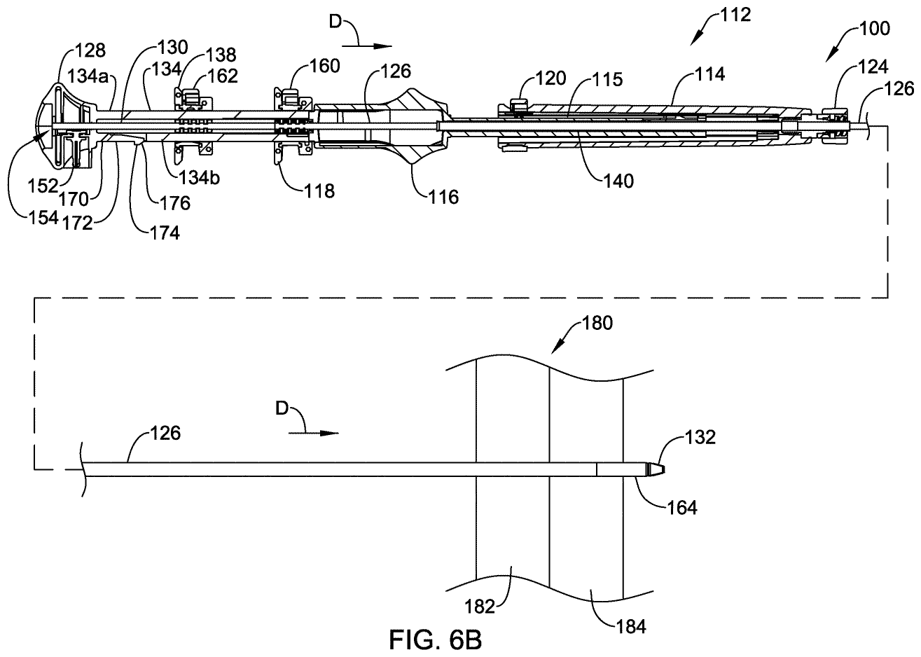

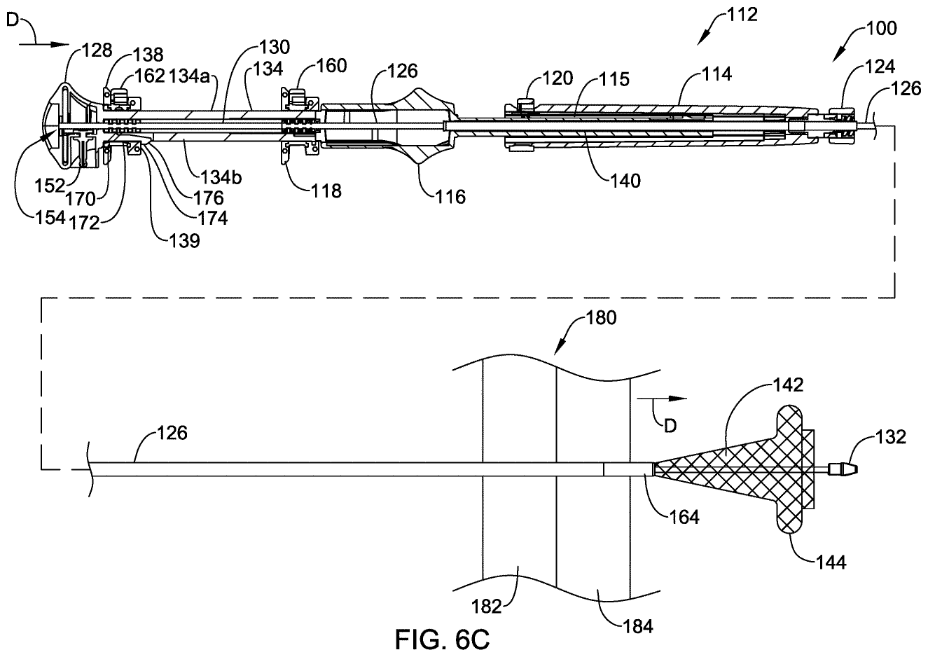

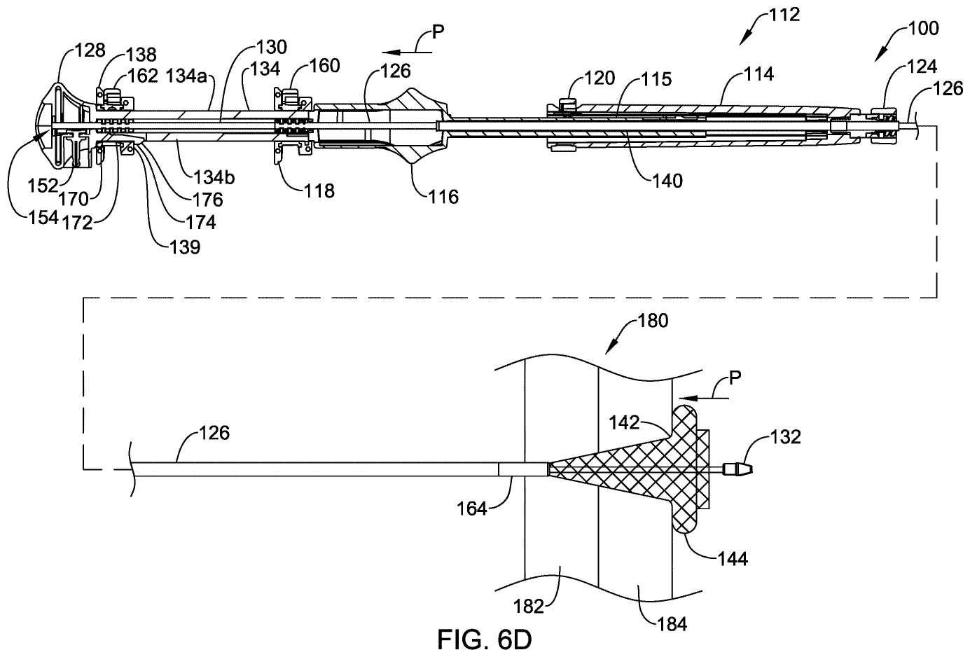

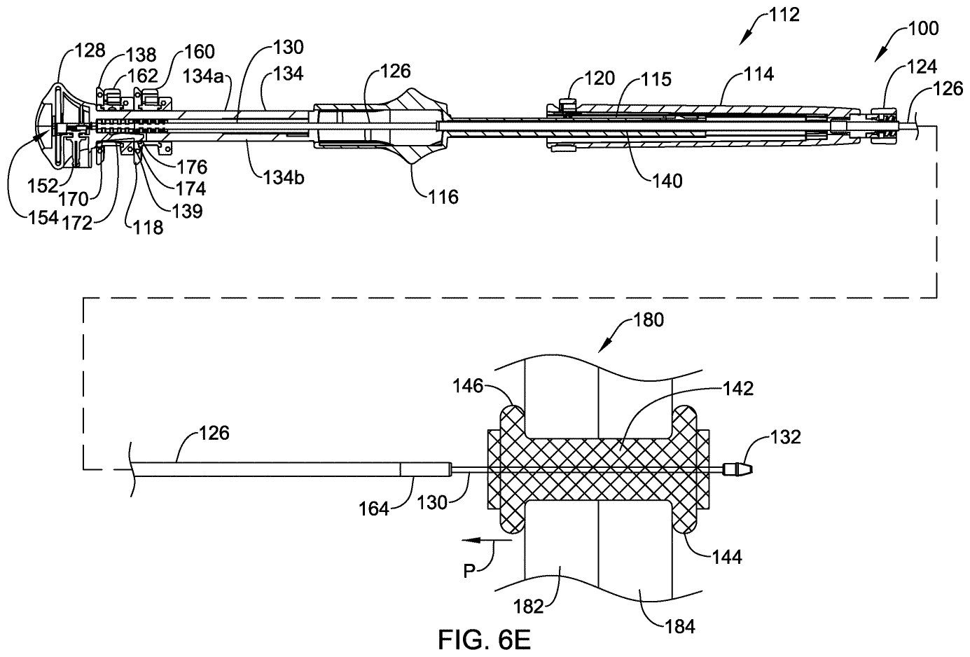

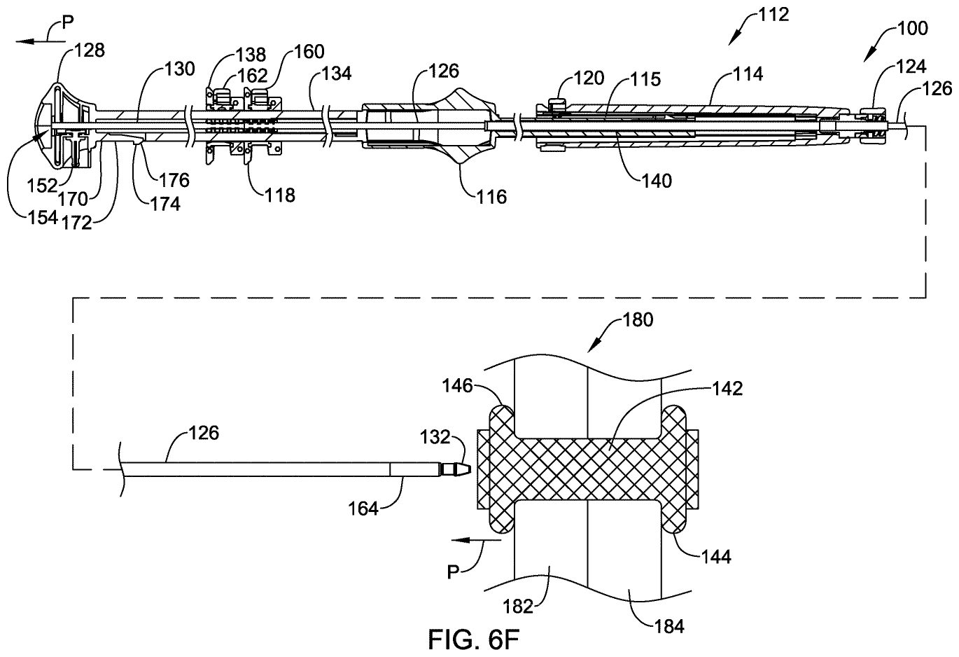

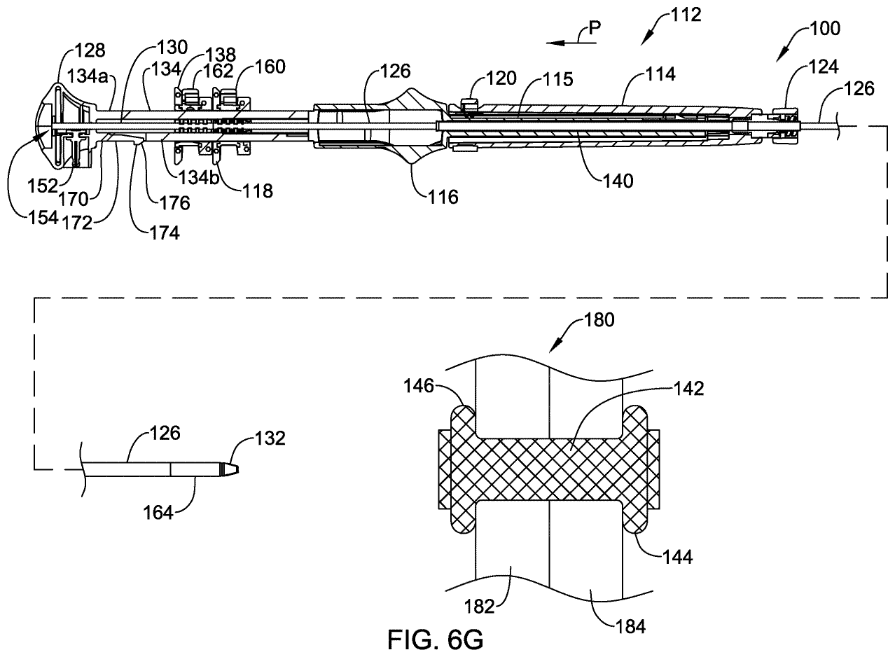

The inner member 130 and/or outer sheath 126 may have one or more visible markers 164 that can be visualized during or after partial retraction of the outer sheath 126. The visible marker(s) 164 may be obscured by tissue walls before extraction. The position of the visible marker(s) 164 on the outer sheath 126 and/or inner member 130 may be an indicator that a stent 142 (FIG. 6C) has been deployed a certain amount. The position of the marker 164 may also be an indicator that the outer sheath 126 has been advanced or retracted to a predetermined position. In some examples, multiple markers 164 may be used on the inner member 130 and/or outer sheath 126 with various colors or patterns that correspond to various positions of the outer sheath 126 and/or the inner member 130. The markers of the outer sheath 126 and/or the inner member 130 may be visible by external imaging, such as x-ray fluoroscopy imaging, directly visible with a scope camera and/or visible by ultrasound.

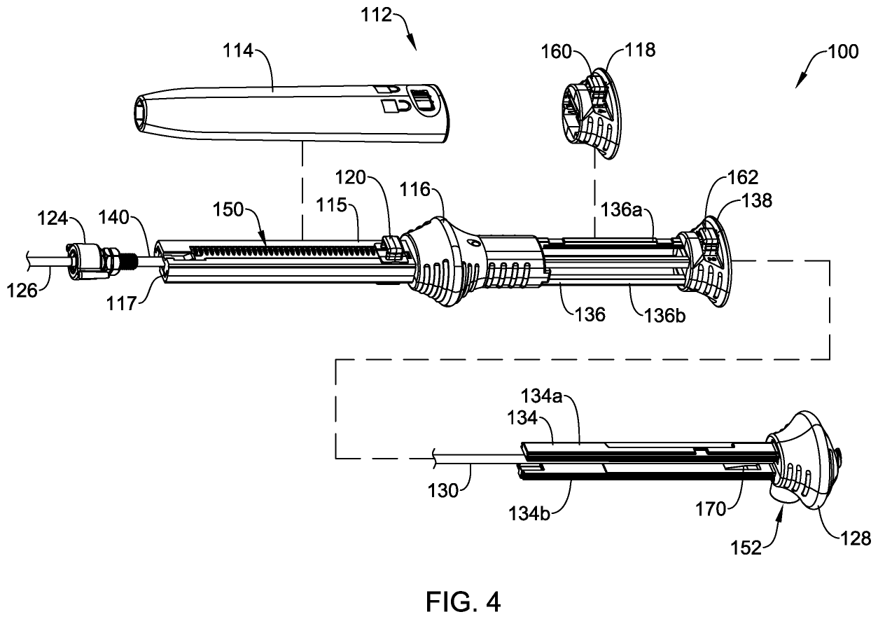

As schematically depicted in the exploded views of FIGS. 3 and 4, respectively, the puller member 118 may be slideably coupled to the outer rails 136. The outer sheath 126 may be attached to the puller member 118 such that movement of the puller member 118 in the proximal direction may cause movement of the outer sheath 126 in the proximal direction without affecting the position of the inner member 130 and distal tip 132. The handle actuator 115 may be coupled to both the inner member 130 and the outer sheath 126, with a handle body sheath 140 (FIG. 3) surrounding both the inner member 130 and outer sheath 126. The stopper 138 may be fixedly attached to the outer rails 136 and configured to limit movement of the puller member 118 (e.g., stop the puller member 118 from being displaced past a certain point, which may be a pre-determined position along the delivery device 100 between the puller member 118 and pusher member 128). The puller member 118 may be advanced in the proximal direction until the puller member 118 reaches the stopper 138. The puller member 118 may include the lock 160 configured to lock and/or unlock the puller member 118 and/or outer sheath 126 from a position relative to the handle actuator 115. When unlocked, the puller member 118 is configured to slide along handle actuator rails 117 (FIG. 4) advancing the attached outer sheath 126 along with the puller member 118.

When the lock 120 is not engaged and the lock 162 of the stopper 138 is engaged, the handle actuator 115 may be adjusted or advanced relative to the handle body 114, moving both the inner member 130 and the outer sheath 126 together. Movement of the handle actuator 115 may adjust the effective working length of the outer sheath 126 and the inner member 130, by adjusting how far the outer sheath 126 and the inner member 130 extend from the distal end of the handle body 114 and consequently how far the inner member 130 and outer sheath 126 extend beyond the distal end of a working channel of an instrument (e.g., scope) through which the delivery device 100 may be placed into the body of a patient. The handle actuator 115 may includes the knob 116 for a user to grip when pushing or pulling the handle actuator 115. The handle actuator 115 may include a lock 120, that may be configured to lock the handle actuator 115 relative to the handle body 114 and relative to the instrument to which the handle body 114 may be attached with the scope lock 124. The handle actuator 115 may include markings 150 (FIG. 4) to illustrate to a physician how far the handle actuator 115 has moved relative to the handle body 114.

The markings 150 may be any suitable type of markings. In some examples, the markings 150 may be grooves or may be printed or stamped on the handle actuator 115. In some examples, the markings 150 may be raised ribs to interfere with the lock 120 to prevent the handle actuator from moving when the lock 120 is engaged with the ribs. The markings 150 may be or may include an indicator corresponding to a predetermined position of the handle actuator 115 relative to the handle body 114.

The handle 112 may include a connector 170 (e.g., a lock mechanism) configured to releasably couple with the stopper 138. In some examples, the connector 170 may be coupled with or extend from the pusher member 128, the stopper 138, and/or the inner rails 134. In operation, the connector 170 may releasably couple the pusher member 128 with the stopper 138 when the pusher member 128 is in a fully advanced position proximate the stopper 138. In some examples, the puller member 118 may fully advance to a position proximate the stopper 138 and disengage the connector 170 (e.g., from the stopper 138) to release the connector 170 and allow relative adjustment between the pusher member 128 and the stopper 138. This configuration of the connector 170 relative to the pusher member 128, the puller member 118, and the stopper 138 may ensure that once the distal flange of the stent 142 is released, the pusher member 128 cannot be advanced in the proximal direction prior to the proximal flange of the stent 142 being released, (e.g. see FIGS. 6A-6E). Other suitable configurations are contemplated for engaging and/or disengaging the connector 170 relative to the stopper 138.

The connector 170 may have any suitable configuration. For example, the connector 170 may be or may include a protrusion, a recess, a tab, a lever, an arm, a leg, a snap, a clip, a switch, a spring (e.g., a metal helical spring, etc.), a spring with a detent mechanism, magnetic lock, a linear tab and slot configured to receive the tab, and/or other suitable mechanism configured to releasably fix the pusher member 128 in a desired position. In one example, the connector 170 may have an arm extending in a distal direction from the pusher member 128 (e.g., from a base of the pusher member) and a tab extending laterally from the arm such that the arm may deflect when the connector 170 reaches or engages the stopper 138 and the tab may engage a surface of the stopper to prevent or mitigate advancement of the pusher member 128 in the proximal direction. In some examples, the connector 170 may be configured to provide audible and/or tactile feed back to a user when the connector engages and/or is disengaged from the stopper 138 and/or other suitable component of the handle 112. Although the connector 170 is depicted as implemented on the pusher member 128 and configured to engage the stopper 138, the connector 170 may be at least partially or entirely implemented on or at the stopper 138 and configured to engage the pusher member 128.

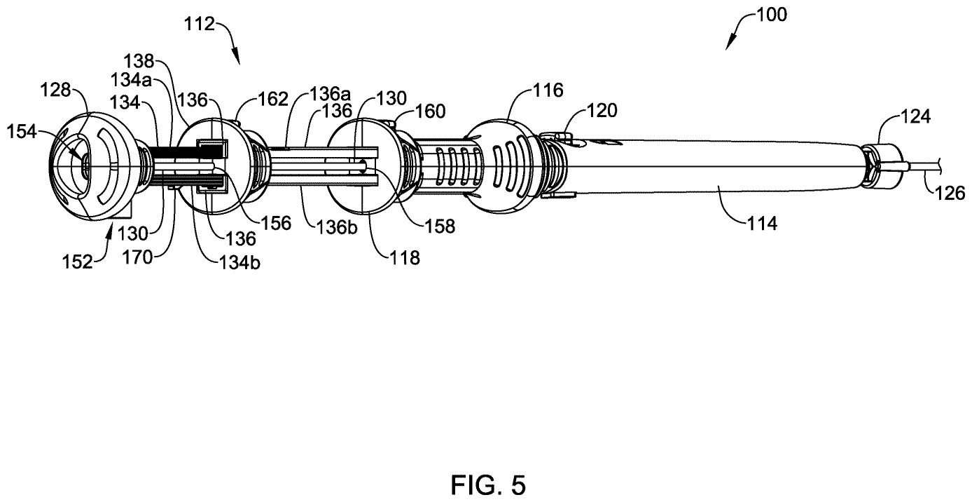

FIG. 5 depicts a schematic perspective proximal end view of the delivery device 100. As shown in FIG. 5, the pusher member 128 may include the electrical plug 152. In some examples, the plug 152 may be configured to deliver electricity to the distal tip 132 of the delivery device 100 in the form of RF energy, HF energy, and/or other suitable type of energy. The inner member 130 may include a lumen 154. The lumen 154 may extend longitudinally through the length of the inner member 130. In some examples, the lumen 154 may be configured to receive a guidewire, fluid, or other component that may be advanced through the lumen 154 to the distal end of the inner member 130.

The pusher member 128 may include a lock (e.g., a luer lock or the like) that may be configured to allow a user to attach a syringe, fluid pump, or the like to the pusher member 128, and flush or inject a fluid, or aspirate a fluid or tissue sample, or the like, through the lumen 154. The syringe or other suitable component attached to the lock may be in fluid communication with the lumen 154 of the inner member 130 allowing the distal end of the inner member 130 to be accessed by a user through the proximal surface of the pusher member 128.

The stopper 138 may include one or more through-channels configured to align the inner rails 134 of the pusher member 128 to the outer rails 136 of the handle actuator 115. The through-channels may be sized and shaped to slideably receive the inner rails 134, allowing the pusher member 128 with inner member 130 attached to advance or slide in the proximal and distal directions. In some examples, the inner rails 134 may include notches that prevent the pusher member 128 from being pulled in the proximal direction past a certain point. The notches, when included, may prevent the inner rails from being pulled through the stopper 138 in the proximal direction.

The puller member 118 may include through-channels that allow the inner rails to pass through the puller member 118. The through channels of the puller member 118 may be sized and shaped to slidably receive the outer rails 136, such that the puller member 118 may slide along and be guided by the outer rails 136 when advanced in the distal direction and/or the proximal direction. The stopper 138 may include a lumen 156 that the inner member 130 can pass through. The puller member 118 may further include a lumen 158 that the inner member 130 may pass through.

FIGS. 6A-6E depict schematic views of steps in an illustrative method of using a delivery device. As demonstrated, for example, in FIGS. 6A-6F, a stent may be deployed to create a channel or opening between two tissue walls. Stent design and placement may be adjusted to accommodate the unique anatomy of a patient and/or area of the body.

Although the method is described with respect to the illustrative delivery device 100 described herein, the delivery device 10 and/or other suitable delivery devices may be utilized. The delivery device configurations of this disclosure may be applied to a variety of procedures in various locations within the body.

FIG. 6A depicts a schematic partial cross-section view of the delivery device 100 with the handle 112 shown in cross-section and the distal end of the delivery device 100 shown in a side view. As depicted in FIG. 6A, the distal tip 132 may be located or positioned at a location proximate body organs or lumen walls 180 having a first lumen wall 182 (e.g., a first tissue wall) defining a first lumen and a second lumen wall 184 (e.g., a second tissue wall) defining a second lumen, with the first lumen wall 182 apposed the second lumen wall 184 (e.g., the distal tip 132 may be positioned at a location proximate a target location). Although the first lumen wall 182 and the second lumen wall 184 are depicted as being in contact with one another prior to a placement of the stent 142, the first lumen wall 182 and the second lumen wall 184 may not be in direct contact prior to placement of the stent 142 and in some examples may not be in direct contact after placement of the stent 142 (e.g., there may be intervening body tissue, etc. between the first lumen wall 182 and the second lumen wall 184 after placement of the stent 142). In some examples, the delivery device 100 may carry the stent 142 (see FIG. 6C) between the inner member 130 and the outer sheath 126.

Although two lumen walls are apposed to one another in FIG. 6A, it is contemplated that more than two walls may be apposed one another. In some examples, the body organs or lumen walls 180 may be a part of the urinary tract, pulmonary tract, gastrointestinal tract, biliary tract, vasculature, or other suitable parts of the body. In some examples, to access a pseudocyst within the second lumen defined by the second lumen wall 184 for drainage of the cyst into stomach (e.g., the first lumen) defined by the first lumen wall 182, a stent may be placed in an opening through the apposed first lumen wall 182 and the second lumen wall 184. Alternatively or additionally, a stent may be placed across a single wall of a single organ or lumen or across a wall shared by two body organs or lumens.

As shown in FIG. 6A, the pusher member 128 may be extended to its most proximal position, the puller member 118 may be in its most distal position, and the handle actuator 115 may be fully extended out of the handle body 114. With the handle 112 in this configuration, the distal tip 132 may be extending out from the distal end of the outer sheath 126, with the inner member 130 still fully within the outer sheath 126. The locks 120, 160, 162 may be engaged (e.g., in a locked configuration) to hold the pusher member 128, puller member 118, and handle actuator 115 in a desired longitudinal location. One or more of the locks 120, 160, 162 may be released (e.g., adjusted to an unlocked configuration) when it is desirable to adjust the position of any of the elements of the device as discussed herein. A guidewire or needle may be advanced through the lumen 154 of the inner member 130 and through the tissue walls prior to passing the distal tip 132 through the first and second lumen walls 182, 184.

As depicted in FIG. 6A, the connector 170 may be configured as including an arm 172 or extending from the pusher member 128 (e.g., extending from a base of the pusher member 128) in a distal direction. In some examples, a protrusion or tab 174 may extend laterally from the arm 172. In some examples, the arm 172 and/or the tab 174 may have a beveled surface 176 (e.g., a tapering distal surface). Although the arm 172 may have other suitable configurations, the arm 172 may be cantilevered from the pusher member 128 and configured to flex or bend laterally from a relaxed or biased first position to a stressed second position (e.g., relative to the base of the pusher member 128) in response to engaging the stopper 138. In some examples, the arm 172 and/or the tab 174 may be configured to automatically engage the stopper 138 and prevent proximal movement of the pusher member 128 when the pusher member 128 is fully advanced in the distal direction.

The lock 120 may be unlocked, which may allow the handle actuator 115 to be adjusted in a distal direction D, as depicted for example in FIG. 6B,. The inner member 130, the outer sheath 126, and the distal tip 132 may move together with the handle actuator 115, the same distance and direction as the handle actuator 115 because the pusher member 128 and inner member 130 are locked against movement relative to the handle actuator 115 by the engaged lock 162 of the stopper 138 and the puller member 118 and outer sheath 126 may be locked against movement relative to the handle actuator 115 by the engaged lock 160 on the puller member 118.

The distal tip 132 may be used to cut, cauterize, or ablate portions of the first lumen wall 182 and/or the second lumen wall 184 to create an opening through the body or lumen walls 182 to allow for easier passage of components of the delivery device 100. In some examples, the distal tip 132 may be configured with a smooth, blunt end so as present an atraumatic surface, reducing the potential of causing damage to the tissue walls. The distal tip 132 may be configured to widen a preexisting opening in the first lumen wall 182 and/or the second lumen wall 184 or create a new opening through the first lumen wall 182 and/or the second lumen wall 184. In some examples, the distal tip 132 may be energized and passed through first lumen wall 182 and the second lumen wall 184. A variety of distal tip 132 designs or configurations may be used with various configurations of the delivery device 100 to access, cut, ablate, or cauterize tissue walls.

After moving the handle actuator 115, the lock 120 may be engaged (e.g., adjusted to a second or locked configuration) to stop movement of the handle actuator 115 relative to the handle body 114. With the lock 120 engaged and the lock 162 at the stopper 138 disengaged (e.g., in an unlocked configuration), FIG. 6C schematically depicts the pusher member 128 being moved in the distal direction D until reaching the stopper 138 (e.g., until the pusher member 128 is fully advanced) to deploy a distal end of the stent 142 from the delivery device 100.

The distal tip 132 and inner member 130 attached to the pusher member 128 may have been moved the same direction and distance as the pusher member 128, as depicted in FIG. 6C (the outer sheath, distal tip 132, and the stent 142 are magnified relative to the handle 112 of the delivery device 100). As the inner member 130 extends past the distal end of the outer sheath 126, a distal end of the stent 142 carried by the delivery device 100 and constrained on the inner member 130 by outer sheath 126 may be partially exposed and partially deployed to an unconstrained configuration. The stent 142 includes a distal retention member 144 (e.g., a distal lobe) on the distal end of the stent 142 which may automatically form when the stent 142 is at least partially exposed. The predetermined position of the stopper 138 along the delivery device 100, the stent 142 on the inner member 130, and a length of the rails 134 and 136 may be calibrated to allow only the distal retention member 144 to be released when the pusher member 128 is moved to the stopper 138. Advancing the distal tip 132 in the distal direction D to deploy the distal end of the stent 142 may result in the distal retention member 144 remaining at a desired location within the patient relative to when the distal end of the stent 142 is deployed by withdrawing the outer sheath 126.

With the pusher member 128 fully advanced in the distal direction D to the stopper 138, the connector 170 may engage the stopper 138 and prevent or mitigate proximal movement of the pusher member 128 relative to the stopper 138 (e.g., prevent or mitigate movement away from the stopper 138). For example, as the pusher member 128 is advanced in the distal direction, the beveled surface 176 may engage the stopper 138 and cause the arm 172 of the connector 170 to deflect to allow the connector to pass at least a portion of the stopper 138. With the pusher member 128 in the fully advanced position, the tab 174 of the connector 170 may engage a distal-facing surface 139 of the stopper 138. In some examples, the distal facing surface 139 of the stopper 138 may be a distal-most surface of the stopper 138, but other suitable locations of the distal-facing surface 139 are contemplated. Other suitable configurations of the connector 170 and the stopper 138 configured to prevent or mitigate proximal movement of the pusher member 128 are contemplated.

The length of the rails 134 and 136 may be calibrated to accommodate a variety of different shapes and sizes of the stent 142. For example, deployment of a stent with an increased length may require longer rails 134 and 136 to fully deploy the proximal and distal retention members 144, 146.

After the distal end of the stent 142 is released from its constraint within the outer sheath 126 by distal movement of the inner member 130 relative to the stationary outer sheath 126, the user may pause to verify the expansion of the distal retention member 144. In some examples, ultrasound imaging may be used to visualize the position of the stent 142 and expansion of the distal retention member 144. In some examples, direct visualization of the stent 142 and its expansion may be possible. The stopper lock 162 may then be engaged to prevent further movement of the pusher member 128 and inner member 130 relative to the stopper 138.

With the lock 162 at the stopper 138 and the lock 160 at the puller member 118 engaged (e.g., in the locked configuration), the lock 120 of the handle actuator 115 may be disengaged (e.g., may be in the unlocked configuration) and the handle actuator 115 may be pulled in a proximal direction P out of the handle body 114, as depicted in FIG. 6D, by adjusting the knob 116 relative to the handle body 114. Advancing the handle actuator 115 in the proximal direction P may correspond to movement of both the outer sheath 126 and the inner member 130 relative to the handle body 114 and instrument (e.g., endoscope) to which the handle body 114 may be attached via the scope lock 124.

In response to the movement of the outer sheath 126 and the inner member 130 in the proximal direction P, the partially deployed stent 142 may be pulled backward with the outer sheath 126 and the inner member 130 until the distal retention member 144 is pulled snuggly up against the second lumen wall 184. In some examples, the distal retention member 144 of the stent 142 may engage the second lumen wall 184 and prevent the stent 142 from being pulled through the second lumen wall 184 and/or the first tissue wall 182 (s) as the delivery device 100 is advanced in the proximal direction.

The lock 120 may be re-engaged to prevent movement of the handle actuator 115, inner member 130, and the outer sheath 126 relative to the handle body 114 once the distal retention member 144 is engaging or is proximate the second lumen wall 184. The lock 120 of the handle actuator 115 may be configured to stop movement of the handle actuator 115 proximally beyond a predetermined position (e.g., to prevent the handle actuator 115 from being advanced in the proximal direction P completely out of handle body 114). In some examples, the markers 164 along the outer sheath 126 may aid in confirming that the handle actuator 115 has been advanced in the proximal direction P a sufficient distance such that a distal end of the outer sheath 126 may be proximate the first lumen wall 182 to allow the proximal end and proximal retention member 146 of the stent 142 to be properly deployed relative to the first lumen wall 182.

Once the handle actuator 115 has been advanced in the proximal direction a desired location (e.g., as discussed with respect to FIG. 6D), the lock 120 may be reengaged (e.g., adjusted to a locked configuration) and the puller member lock 160 may be disengaged (e.g., adjusted an unlocked configuration), allowing puller member 118 to be advanced in the proximal direction P along with attached outer sheath 126, and relative to a stationary inner member 130, as depicted for example in FIG. 6E. With the distal end of the stent 142 deployed, the proximal end of the stent 142 may then be deployed when the outer sheath 126 is advanced in the proximal direction P in response to the user advancing the puller member 118 in the proximal direction P. The puller member 118 may be pulled proximally until the puller member 118 reaches the stopper 138.

As the puller member 118 approaches the stopper 138 and/or engages the stopper 138, the puller member 118 may engage the connector 170 and disengage the connector 170 from the stopper 138. In one example, as the puller member 118 is advanced in the proximal direction P, the puller member 118 (e.g., a proximal end of the puller member 118) may engage the beveled surface 176 of the connector 170 to deflect the arm 172 laterally and disengage the tab 174 from the distal-facing surface 139 of the stopper 138, as depicted for example in FIG. 6E. Other suitable configurations for disengaging the connector 170 from the stopper 138 are contemplated. Once the connector 170 has disengaged from the stopper 138, the pusher member 128 may be free to adjust in the proximal direction P. In this manner, the pusher member 128 may be prevented from advancing in the proximal direction P and causing the inner member 130 and the distal tip 132 to move in the proximal direction P relative to the outer sheath 126 until stent 142 has been fully deployed or deployed to some other desired extent.

The lock 160 of the puller member 118 may be configured to automatically engage (e.g., automatically adjust to the locked configuration) during advancement in the proximal direction P when the outer sheath 126 is retracted to a predetermined position. Alternatively, the lock 160 may be engaged manually. In some examples, the puller member 118 may only be locked by the lock 160 when the puller member 118 is in the distalmost position if the stent 142 is completely undeployed. In other examples, the puller member 118 may be locked at any point during proximal retraction by the lock 160. In some examples, the puller member 118 may be locked against the stopper 138 to prevent further movement of the outer sheath 126 relative to the inner member 130.

The markers 164 on the outer sheath 126 and/or the inner member 130 may be visible when the outer sheath 126, or the delivery device 100 together with the outer sheath 126, has been retracted enough to deploy the proximal retention member 146 of the stent 142 (e.g., the distal end of the outer sheath 126 may be proximal of a proximal-most end of the stent 142). The direct visibility of the marker 164 may signal to the user that the stent 142 has been fully deployed, or that the body organs or lumen walls 180 have been sufficiently traversed by the proximal retention member 146. In other words, direct visibility of the marker 164 may signal to the user that the delivery device 100 and outer sheath 126 have been retracted a sufficient distance proximal of the first lumen wall 182 to allow the proximal end and proximal retention member 146 of the stent 142 to be properly deployed at the first lumen wall 182 (e.g., lumen or organ defined by the first lumen wall 182). Proper placement and/or deployment of the stent 142 may be confirmed by fluoroscopy imaging as well. The proximal retention member 146, along with the distal retention member 144, may prevent migration of the stent 142 through first lumen wall 182 and/or the second lumen wall 184 after deployment of the stent 142 and, when spanning apposed walls, may assist in maintaining the walls 182, 184 in apposition.

As shown in FIG. 6F, when the stent 142 is in its fully deployed configuration, the stent 142 may be sized and shaped such that inner member 130 and the distal tip 132 may be withdrawn proximal of the stent 142. In some examples, with the connector 170 disengaged from the stopper 138, the pusher member 128 may be advanced in the proximal direction P to move the inner member 130 and the distal tip 132 in the proximal direction and position the distal tip 132 at the distal end of the outer sheath 126. In some examples, positioning the distal tip 132 at the distal end of the outer sheath 126 may facilitate visualizing the distal end of the delivery device 100 during withdrawal of the delivery device 100 from the patient and prevent or mitigate the chances of a proximal facing surface of the distal tip 132 catching on tissue within the lumen(s) through which the delivery device 100 may advance in the proximal direction P during withdrawal from the patient.

With the distal tip 132 at the distal end of the outer sheath 126, the distal tip 132, inner member 130, and outer sheath 126 may be withdrawn from the body lumen(s) of the patient, while leaving the stent 142 in place. As depicted in FIG. 6G, the handle body 114 and the knob 116 may be advanced in the proximal direction P to advance the distal tip 132 in the proximal direction P and out of the body lumen(s) of the patient. It is contemplated that other suitable manipulations of the delivery device 100 may utilized to remove the distal tip 132 from the body lumen(s) of the patient.

The distal tip 132 of the delivery device 100 may be visualized as the distal tip 132 is advanced in the proximal direction P (e.g., withdrawn) from the location proximate the target location (e.g., the organ or body lumen walls or other suitable location). Any suitable visualization technique may be utilized including, but not limited, visualization with a scope that may or may not be part of the delivery device 100 or through which the delivery device 100 may travel to the target location.

Although not depicted, the pusher member 128 may be configured as or include a rotatable cam. Example pusher members configured as or including a rotatable cam include, but are not limited to the examples described in U.S. patent application Ser. No. 18/960,832, titled DEVICES, SYSTEMS AND METHODS FOR ACCESS AND DELIVERY TO CHALLENGING ANATOMY, and filed on Nov. 26, 2024, which is hereby incorporated by reference in its entirety for any and all purposes.

In some configurations, the stent 142 may be a self-expanding stent made from a metal, metal alloy, polymer (some examples of which are disclosed below), a metal-polymer composite, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; cobalt chromium alloys, titanium and its alloys, alumina, metals with diamond-like coatings (DLC) or titanium nitride coatings, other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “super-elastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In at least some configurations, portions or all of the stent delivery device 10, 100 (and variations, systems or components thereof disclosed herein) may also be doped with, made of, or otherwise include a radiopaque material. The stent itself, delivery device, or both may include radiopaque markers delineating the ends of the stent for placement or the position of the outer sheath with respect to the inner member The radiopaque markers may also delineate the position of the inner member and/or outer sheath with respect to one or more reference points in the body. Markers such as textured or patterned sections that show up on ultrasound or markers that are directly visible may be utilized as well in various combinations and for different or combined purposes. Radiopaque materials are understood to be materials capable of producing a relatively bright image on a fluoroscopy screen or another imaging technique during a medical procedure. This relatively bright image aids a user in determining the location of the stent delivery device (and variations, systems or components thereof disclosed herein). Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stent delivery device (and variations, systems or components thereof disclosed herein) to achieve the same result.

In some configurations, the stent delivery device 10, 100 (and variations, systems or components thereof disclosed herein) and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, Elast-Eon® from AorTech Biomaterials or ChronoSil® from AdvanSource Biomaterials), biocompatible polymers, other suitable materials, or mixtures, combinations, copolymers thereof, polymer/metal composites, and the like. In some configurations, the sheath can be blended with a liquid crystal polymer (LCP). For example, the mixture can contain up to about 6 percent LCP.

It should be understood that this disclosure is, in many respects, only illustrative. Changes may be made in features and details, particularly in matters of materials, shape, size, and arrangement of steps without exceeding the scope of the disclosure. This may include, to the extent that it is appropriate, the use of any of the features of one example configuration being used in other configurations. The scope of the disclosure is, defined by the language in which the appended claims are expressed.

What is claimed is:

1. A delivery device, the device comprising:
an outer sheath having a proximal end, a distal end, and a lumen extending longitudinally therethrough;
an inner member disposed within the lumen of the outer sheath; and
a handle comprising:
a body;
a puller member coupled with the proximal end of the outer sheath;
a pusher member coupled with the proximal end of the inner member;
a stopper coupled with the body and positioned between the puller member and the pusher member; and
a connector,
wherein adjustment of the pusher member toward the stopper adjusts the inner member relative to the outer sheath and causes the connector to prevent proximal movement of the pusher member relative to the stopper.

2. The delivery device of claim 1, wherein the puller member is configured to be adjusted toward the stopper to adjust the outer sheath relative to the inner member and engage the connector to release the connector from engagement with the stopper.

3. The delivery device of claim 1, wherein the connector comprises a tapering distal surface and the puller member is configured to adjust relative to the stopper to engage the tapering distal surface to release the connector from engagement with the stopper.

4. The delivery device of claim 1, wherein the pusher member has a base and the connector is configured to extend in a distal direction from the base.

5. The delivery device of claim 4, wherein the connector is configured to be biased to a first position and flex to a second position relative to the base.

6. The delivery device of claim 1, wherein the connector comprises a protrusion configured to engage a distal facing surface of the stopper.

7. The delivery device of claim 1, wherein the stopper comprises a lock, the lock is configured to adjust from a first position in which the pusher member is able to adjust longitudinally relative to the stopper and a second position in which the pusher member is longitudinally fixed relative to the stopper.

8. The delivery device of claim 1, wherein the handle further comprises:
an actuator disposed within a lumen of the body,
wherein adjustment of the actuator relative to the body longitudinally adjusts the outer sheath and the inner member relative to the body.

9. The delivery device of claim 1, further comprising:
a distal tip disposed at a distal end of the inner member,
wherein the distal tip is distal of the outer sheath when the inner member is fully advanced in a proximal direction relative to the outer sheath.

10. A system, the system comprising:
a delivery device, comprising:
an outer sheath having a proximal end, a distal end, and a lumen extending longitudinally therethrough;
an inner member disposed within the lumen of the outer sheath; and
a handle comprising:
a body;
a puller member coupled with the proximal end of the outer sheath;
a pusher member coupled with the proximal end of the inner member, the pusher member comprising a connector; and
a stopper coupled with the body and positioned between the puller member and the pusher member; and
a self-expanding stent constrained on the inner member between the inner member and the outer sheath and deployable therefrom by first pushing the inner member with the pusher member relative to the outer sheath and then pulling the outer sheath with the puller member relative to the inner member,
wherein pushing inner member with the pusher member causes the connector to engage the stopper and prevent proximal movement of the inner member relative to the outer sheath.

11. The system of claim 10, wherein the puller member is configured to engage the connector to release the connector from engagement with the stopper and allow proximal movement of the inner member relative to the outer sheath.

12. The system of claim 10, wherein when the self-expanding stent is fully deployed, the self-expanding stent is shaped and sized such that a distal tip of the inner member can be retracted proximally through the self-expanding stent.

13. The system of claim 10, wherein the connector comprises a tapering distal surface and the puller member is configured to engage the tapering distal surface to release the connector from engagement with the stopper.

14. The system of claim 10, wherein the connector comprises a protrusion configured to engage a distal facing surface of the stopper.

15. The system of claim 10, wherein the stopper comprises a lock, the lock is configured to adjust from a first position in which the pusher member is able to adjust longitudinally relative to the stopper and a second position in which the pusher member is longitudinally fixed relative to the stopper.

16. A method of using a stent delivery device, the method comprising:
positioning a distal tip of a delivery device at a location proximate a target location, the delivery device carrying a stent at a location proximate the distal tip and between an inner member and an outer sheath of the delivery device;
deploying a distal end of the stent from the delivery device by advancing a pusher member of the delivery device toward a stopper of the delivery device until a connector of the pusher member couples with the stopper to prevent movement of the pusher member away from the stopper; and
with the distal end of the stent deployed, deploying a proximal end of the stent from the delivery device by advancing a puller member of the delivery device toward the stopper until the puller member engages the connector and causes the connector to disengage from the stopper.

17. The method of claim 16, further comprising:
after the connector is disengaged from the stopper, withdrawing the distal tip of the delivery device to a location proximate a distal end of the outer sheath by advancing the pusher member in a direction away from the stopper.

18. The method of claim 17, further comprising:
with the distal tip at the location proximate the distal end of the outer sheath, withdrawing the distal tip of the delivery device in a proximal direction from the location proximate the target location.

19. The method of claim 18, further comprising:
visualizing the distal tip of the delivery device as the distal tip is withdrawn in the proximal direction from the location proximate the target location.

20. The method of claim 16, wherein positioning the distal tip of the delivery device at the location proximate the target location comprises advancing an actuator of the delivery device relative to a handle body of the delivery device.
